(19) United States
(12) Patent Application Publication
(10) Pub. No.: US 2006/0105388 A1
(43) Pub. Date: May 18, 2006

(54) Incorporation of hapten groups during the production of carriers for the determination of analytes

(75) Inventor: Beier Markus, Heidelberg (DE)

Correspondence Address: ROTHWELL, FIGG, ERNST & MANBECK, P.C., 1425 K STREET, N.W., SUITE 800, WASHINGTON, DC, 20005, US

(73) Assignee: Febit Biothch GmbH, Heidelberg (DE)

(21) Appl. No.: 10/540,392

(22) PCT Filed: Dec. 23, 2003
(86) PCT No.: PCT/EP03/14820

§ 371 Date: Jun. 23, 2005

(30) Foreign Application Priority Data
Dec. 23, 2002 (DE) 102 60 591.2
Dec. 23, 2002 (DE) 102 60 592.0
May 7, 2003 (DE) 103 20 339.7

(51) Int. Cl. G01N 33/53

(52) U.S. Cl. 435/7.1; 435/7.5

(57) ABSTRACT

The invention relates to a method for producing a carrier, especially microfluidic carrier, for the determination of analytes.

The invention relates to a method for producing a carrier, in particular a microfluidic carrier, for the determination of analytes.

In recent years, valuable means permitting rapid and highly parallel performance of complex determinations of analytes have been created with the technology of receptor arrays, e.g. DNA chips, immobilized on a carrier. The biophysical principle underlying the receptor arrays is that of the interaction of a specific immobilized receptor with an analyte present in a liquid phase, for example through nucleic acid hybridization, where a large number of receptors, e.g. hybridization probes, each of which binds specifically to different analytes, e.g. complementary nucleic acid analytes, present in the sample, are applied to different zones of the carrier.

In order to be able to deal with complex biological questions such as gene expression studies, target validation, sequencings or resequencings using receptor arrays, e.g. using DNA chips, efficient production of receptor arrays in high quality is of fundamental importance. DNA arrays can be produced for this purpose not only by spotting technology (Cheung et al., Nature Genet. Suppl. 1999, Vol. 21, 15-19) but also in situ with use of phosphoramidite synthesis building blocks (Caruthers et al., Tetrahedron Lett., 1981, 1859). It is possible in this connection to distinguish between wet chemical methods (Maskos et al., Nucleic Acids Res. 1992, Vol. 20, 1679-1984) and photochemical methods (Pease, Proc. Natl. Acad. Sci., 1994, Vol. 91, 5022-5026).

A carrier and a method for determining analytes which permit integrated receptor synthesis and analysis are described for example in WO 00/13018. The receptor synthesis therein preferably takes place with use of photoactivatable receptor building blocks. Alternatively, synthesis of the receptor building blocks also by wet chemical methods is disclosed. DE 101 22 357.9 describes a method for receptor synthesis which comprises the use of a combination of photochemical and wet chemical steps.

One object of the present invention was to provide a method for producing a carrier for the determination of analytes which makes it possible to control the synthesis of receptors on the carrier efficiently.

The object is achieved by a method in which hapten groups are applied to the carrier before the process for synthesizing or/and during one or more steps of the process for synthesizing receptors.

A first aspect of the invention is a method for producing a carrier for the determination of analytes, comprising the steps:
(a) providing a carrier,
(b) passing liquid with building blocks for synthesizing polymeric receptors over the carrier,
(c) site- or/and time-specifically immobilizing the receptor building blocks on respective predetermined zones on the carrier and
(d) repeating steps (b) and (c) until the desired receptors have been synthesized on the respective predetermined zones,
   characterized in that hapten groups are applied to the carrier before, during or/and after the synthesis of the receptors.

A second aspect of the invention is a method for quality control of receptor syntheses on a carrier, comprising the steps;
(a) providing a carrier,
(b) applying in planar fashion hapten groups to the carrier surface,
(c) carrying out a receptor synthesis on the carrier,
(d) contacting with a hapten detection reagent which permits detection of hapten groups,
(e) evaluating the hapten group detection on the carrier and
(f) correlating the result of the evaluation with the quality or/and efficiency of the receptor synthesis.

A third aspect of the invention is a method for quality control of receptor syntheses, comprising the steps:
(a) providing a carrier,
(b) carrying out a receptor synthesis on the carrier, with hapten groups being incorporated during the synthesis into the receptor molecules at pre-determined positions,
(c) contacting with a hapten detection reagent which permits detection of hapten groups,
(d) evaluating the hapten group detection on the carrier and
(e) correlating the result of the evaluation with the quality or/and efficiency of the receptor synthesis.

The present invention is particularly distinguished in that the method for producing the carrier can be integrated with a detection system for the determination of analytes. This detection system can be employed for integrated synthesis and analysis, in particular for assembling complex carriers, e.g. bio-chips, and for analyzing complex samples, e.g. for genomic, gene expression or proteomic analysis.

The receptors are synthesized in situ on the carrier, for example by passing fluid with receptor synthesis building blocks over the carrier, immobilizing the building blocks in a site- or/and time-specific fashion on the respective predetermined zones on the carrier, and repeating these steps until the desired receptors have been synthesized on the respective predetermined zones on the carrier.

An essential feature of the method of the invention is that hapten groups are applied to the carrier in a planar or/and spatially resolved fashion before, during or/and at the end of a spacer assembly taking place before the actual receptor assembly, or/and before, during or/and at the end of the receptor synthesis.

The carrier produced by the method of the invention is preferably integrated in a device for determining analytes, comprising
(i) a light source matrix, preferably a programmable light source matrix, e.g. selected from a light valve matrix, a mirror array, a UV laser array and an LED array,
(ii) a carrier, preferably a microfluidic carrier with channels, in particular with closed channels in which the predetermined zones with the respective differently immobilized receptors are located, where the channels preferably lie within the range from 10 μm to 10000 μm, particularly preferably in the range from 50 to 250 μm, and can in principle be designed in any form, e.g. with round, oval, square or rectangular cross section,
(iii) means for supplying fluid to the carrier and for discharging fluid from the carrier, and
(iv) a detection matrix, e.g. an optical detection matrix, such as, for example, a CCD matrix or/and an electronic detection matrix as described in WO 00/13018.

In a preferred embodiment, the carrier provides, through division into fluidic subspaces which can be addressed separately from one another, the possibility of determining the site-specific immobilization. A carrier which satisfies this criterion is described in WO 00/13018. The carrier in this case provides for division of the reactive zones into 2 or more subspaces.

The receptors are preferably selected from biopolymers which can be synthesized in situ on the carrier from the appropriate synthesis building blocks by a combination of light-controlled and wet chemical processes. Synthesis building blocks which can be employed in this connection are both monomeric, e.g. mononucleotides, amino acids etc., and oligomeric building blocks, e.g. di-, tri- or tetranucleotides, di-, tri- or tetrapeptides etc. The receptors are preferably selected from nucleic acids such as DNA, RNA, nucleic acid analogs such as peptide nucleic acids (PNA), proteins, peptides and carbohydrates. The receptors are particularly preferably selected from nucleic acids and nucleic acid analogs and are employed in a detection method for hybridization of complementary nucleic acid analytes. The receptors are immobilized on the surface preferably via spacer groups. Spacer groups can also be assembled stepwise from appropriate synthesis building blocks.

The spacer or receptor synthesis preferably comprises the use of synthesis building blocks with wet chemical protective groups or/and of receptor building blocks with photochemical protective groups. It is also possible where appropriate to use synthesis building blocks which have both wet chemical and photochemical protective groups or hybrid protective groups, i.e. groups which can be eliminated in two stages by a wet chemical and a photochemical step. Examples of wet chemical protective groups are any protective groups disclosed in the prior art for synthesizing biopolymers such as, for example, nucleic acids or peptides on solid carriers. Preferred examples are acid-labile protective groups, base-labile protective groups, oxidation-labile protective groups or protective groups which can be eliminated enzymatically. The use of acid-labile protective groups such as, for example, dimethoxytrityl is particularly preferred. The photo-chemical protective groups which can be employed for the photochemical synthesis steps are any of those disclosed in the prior art for synthesizing biopolymers such as, for example, nucleic acids or peptides on solid carriers. Preferred examples of photochemical protective groups are described in DE 101 05 079.8 and preferred examples of hybrid protective groups are described in DE 101 05 077.1. It is additionally possible also to use two-stage protective groups requiring a photochemical and a wet chemical step for elimination as described in DE 101 32 025.6.

The method of the invention preferably comprises the production of a carrier with a plurality, preferably with at least 50 and particularly preferably with at least 100 different receptor zones which are able to react with respective different analytes in a single sample. The method of the invention can be employed to produce carriers where the receptors in each zone of the carrier comprise only a single sequence of building blocks. In a further embodiment, the method of the invention can, however, also be employed to produce carriers where the receptors in at least one zone of the carrier comprise a plurality of different sequences of building blocks.

The method of the invention comprises the application of hapten groups to the carrier used to produce receptors. These hapten groups are preferably selected from organic molecules having a molecular weight of up to 2000, in particular up to 1000, which are recognized by a specific binding partner, e.g. a protein such as, for example, an antibody, streptavidin, avidin or a lectin, through a high-affinity interaction. The term “high-affinity interaction” means in this connection that the interaction between hapten group and binding partner is sufficiently strong to enable the incorporation of hapten groups on the carrier to be controlled under particular operating conditions with an appropriate detection reagent. Preferred examples of haptens are digoxin and digoxigenin, and dinitrophenol (DNP), which are recognized by appropriate antibodies, and biotin or biotin analogs such as, for example, iminobiotin, aminobiotin or dethiobiotin, which are recognized by streptavidin and avidin.

The hapten groups can be applied in planar or site-specific fashion to the carrier. Combinations of application in planar fashion and application in site-specific fashion, for example with use of two or more different hapten groups, are also possible. In this connection, “application in planar fashion” means that hapten groups are applied to the complete surface of the carrier or a part thereof which comprises zones for receptor synthesis and adjacent zones on which no receptor synthesis is to take place. On the other hand, “application in site-specific fashion” means that the hapten groups are applied selectively onto respective single zones or groups of zones for the receptor synthesis.

In a preferred embodiment, the hapten groups are inserted into a spacer. Such a spacer is disposed between the actual carrier surface and the receptor and can be used to adjust the distance of the receptor from the surface, or the receptor density within the predetermined zones, to an optimal value. The spacers are preferably likewise synthesized by site- or/and time-specifically immobilizing individual spacer building blocks until the desired spacers have been synthesized on the respective predetermined zones. The chemistry of spacer assembly can take place in analogy to the chemistry of receptor synthesis with use of analogous building blocks which, however, comprise no receptor elements, e.g. through use of phosphoramidite building blocks without nucleobases.

The derivatization of the carrier or/and the surface density of the spacer molecules can be determined by incorporating hapten groups into the spacer. Thus, for example, a hapten group can be incorporated into the spacer during the spacer assembly, and planar staining of the carrier surface by the specific binding partner of the hapten group can take place during or/and after the spacer assembly. The signal obtained with this staining permits conclusions to be drawn about the quality of the carrier derivatization and makes it possible to determine whether the carrier can in fact be used for later receptor synthesis. One advantage of this embodiment is that no incorporation of the hapten group into the receptor takes place, and the hapten group cannot have interfering effects either in the following receptor synthesis or in an analyte determination.

In yet a further embodiment, the hapten group can be applied in planar fashion to the complete carrier surface. Then a receptor synthesis, where appropriate together with a spacer synthesis, is carried out by a known method, and subsequently the surface of the carrier is stained by the hapten binding partner. No staining by the binding partner can take place on zones where successful receptor synthesis has taken place. The degree of absence of a staining correlates with the receptor density. This procedure also has the advantage that the receptors used for determination of analytes have a hapten group, and the hapten group cannot have interfering effects in the subsequent determination of analytes.

In yet a further embodiment of the present invention, the hapten groups are inserted at one or more positions into the receptors synthesized on the carrier, e.g. at the start, in the middle or at the end of the receptor. This procedure permits the efficiency of receptor synthesis to be controlled via the number of hapten groups inserted into a zone.

The hapten groups can be inserted reversibly or irreversibly. A reversible insertion of hapten groups has the advantage that the groups can be eliminated again at defined times during or after the receptor synthesis, and thus cannot lead to an impairment of analyte binding to the receptor.

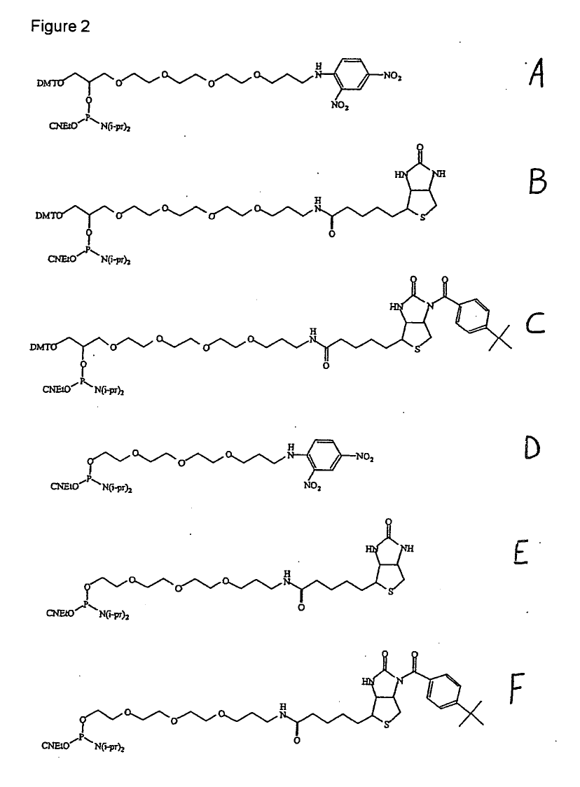

The different modes of inserting hapten groups make reversible and irreversible monitoring possible. Reversible monitoring means that incorporation of such reagents can be followed by further chain assembly at this position, i.e. these reagents are suitable not only for terminal but also for internal use/quality control of receptor synthesis (Example FIG. 2A, B, C). Irreversible monitoring means that incorporation of such reagents cannot be followed by further chain assembly at this position, i.e. these reagents are suitable for terminal use/quality control of receptor synthesis (Example FIG. 2 D, E, F).

The present invention is additionally to be illustrated by the following figures:

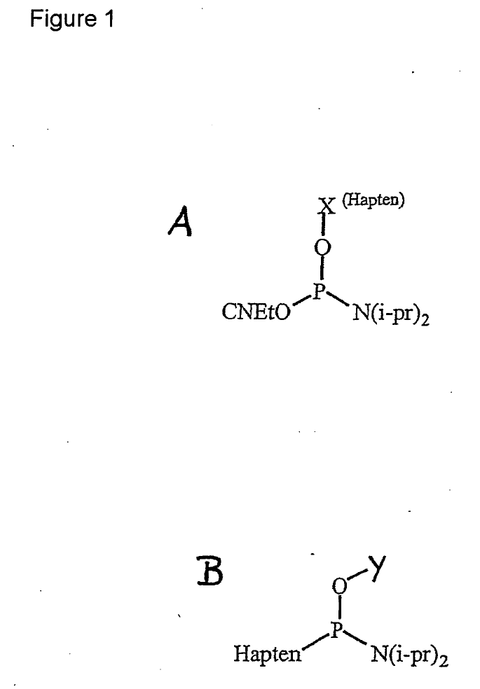

FIG. 1 shows two embodiments of hapten group-containing synthons which can be employed to produce receptors or/and spacers. The synthons are phosphoramidites. It is, however, also possible to employ other synthon types correspondingly.

FIG. 1A shows a phosphoramidite building block which is substituted by a diisopropylamino group, a cyano-ethoxy group and a further functionality X which carries a hapten group. The functionality X may comprise for example a nucleobase or/and a spacer unit.

FIG. 1B shows a phosphoramidite building block which is modified with a diisopropylamino group, a hapten group which is linked where appropriate via a spacer to the phosphorus atom, and a group O-Y where Y may comprise a spacer group or a nucleobase. Here, in contrast to FIG. 1A, the hapten function is linked directly to the phosphitamide unit.

FIG. 2 depicts specific embodiments of the phosphoramidite derivative shown in FIG. 1A. The hapten group in the derivatives shown in FIG. 2A and D is a dinitrophenyl group. The hapten group in the derivatives shown in FIG. 2B and E is a biotin group. The hapten group in the derivatives shown in FIG. 2C and F is a biotin group protected by a tert-butylbenzoic acid group. FIGS. 2A-C show derivatives which additionally have a DMT (dimethoxytrityl group). This has the advantage that, after coupling with this reagent, the oligonucleotide synthesis can be continued after abstraction of the terminal DMT group. These reagents are thus additionally suitable for internal labeling of an oligonucleotide with haptens, whereas the reagents in FIG. 2D-E are for terminal hapten labeling.

The derivatives shown in FIG. 2A-C are suitable for reversible monitoring, while the derivatives shown in FIG. 2D-F can be employed for irreversible monitoring.

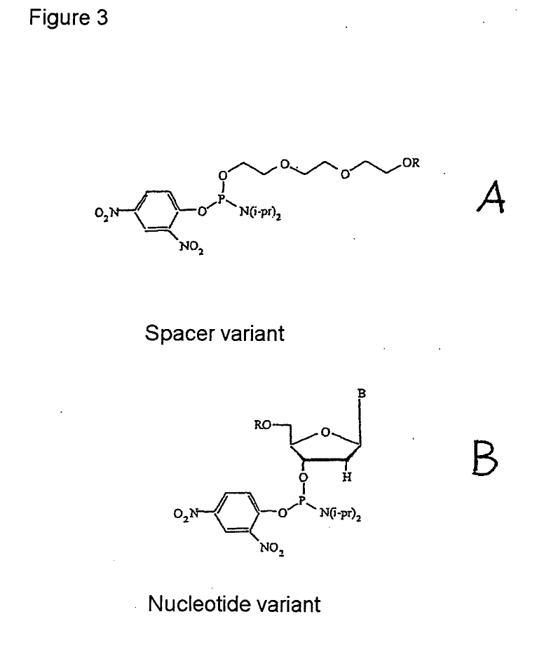

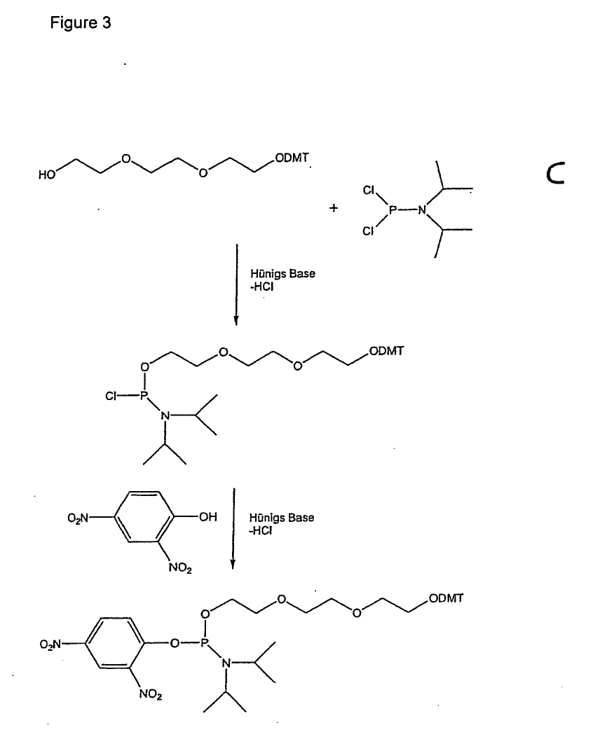

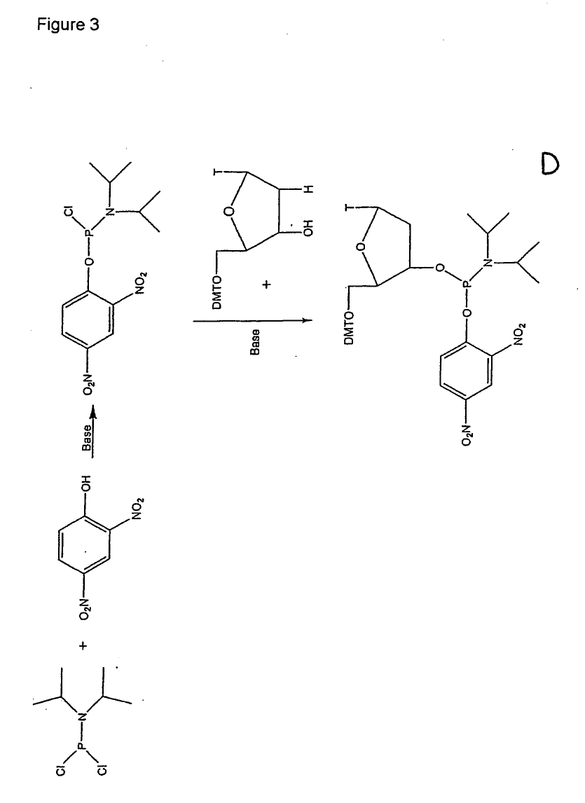

FIG. 3 shows specific examples of the derivative depicted in FIG. 1B. A dinitrophenyl group is present as hapten group. In the variant shown in FIG. 3A, the group Y is a spacer group, where R depicts a protective group, e.g. a photolabile or acid-labile protective group. In the variant shown in FIG. 3B, Y is a nucleoside group comprising a deoxyribose unit and nucleobase (B), where the 5′-position of the deoxyribose unit is blocked by a protective group R. Both reagents are additionally suitable for internal labeling of an oligonucleotide with haptens.

The compounds shown in FIG. 3A are produced in accordance with the synthesis scheme in FIG. 3C. The compound shown in FIG. 3B is produced according to the synthesis scheme in FIG. 3D, with use for example of T as nucleobase.

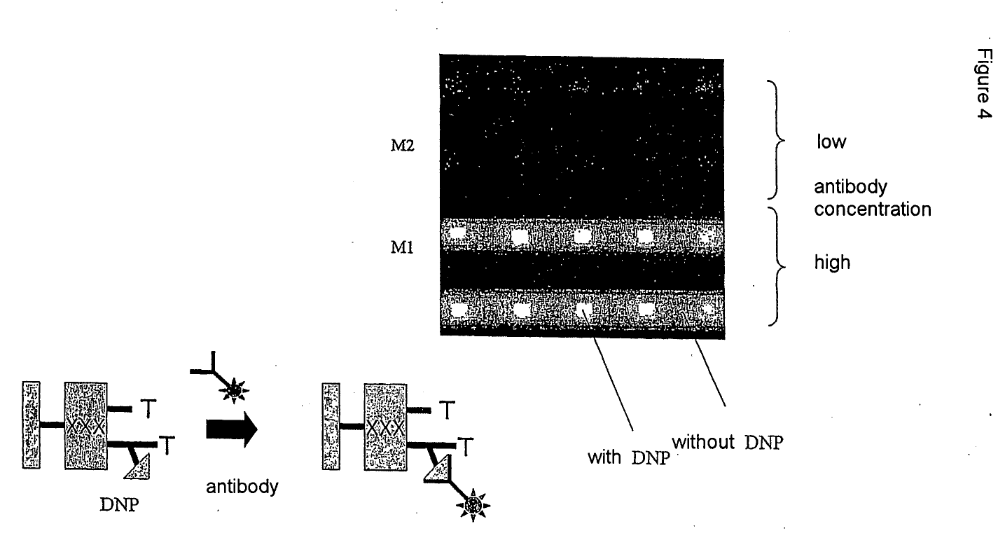

FIG. 4 shows a spatially resolved incorporation of the hapten DNP on a carrier at zones for receptor synthesis and detection thereof using an anti-DNP antibody (labeled with the fluorescent dye Alexa 488).

After the coupling in planar fashion of two spacer units (X), a DNP group (DNP) was applied in a spatially resolved fashion. This was followed by application in planar fashion of a thymidine unit (T).

Detection took place using an anti-DNP antibody which was labeled with Alexa-488. The only positions to emit a signal are those on which a DNP unit was condensed. The signal strength depends on the antibody concentration.

A building block of the type of FIG. 2A was used as DNP group, i.e. a reversible monitoring was carried out. After abstraction of the DMT group present on the DNP unit it was possible to continue the receptor synthesis.

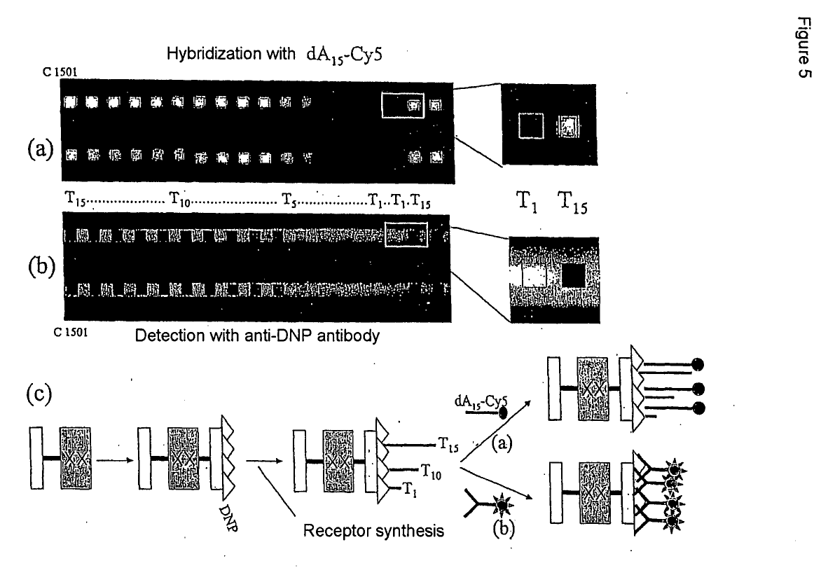

FIG. 5 shows the coupling in planar fashion of two spacer units (X) and the subsequent application in planar fashion of DNP onto the carrier before a spatially resolved receptor synthesis of homomeric thymidine units.

(a) Hybridization with a Cy5-labeled complementary dA-15 sequence was carried out. Hybridizations on the complementary thymidine sequences (from T₁₅ . . . T₁) are evident with decreasing signal intensity.

(b) Detection was carried out with an anti-DNP antibody labeled with Alexa-488.

A signal is emitted in this case in planar fashion and derives from the presence of DNP units which is interrupted only at the points where thymidine units were assembled (negative signal). In addition the strength of this negative signal increases with the length of the thymidine sequence. The length of the receptor, i.e. the success of a synthesis, can be detected through an increasing negative signal (decreasing positive signal) (negative correlation of synthesis and detection signal).

The DNP group used was a building block of the type shown in FIG. 2A, i.e. a reversible monitoring was carried out. After abstraction of the DMT group present on the DNP unit it was possible to continue the receptor synthesis.

(c) A diagrammatic representation of the processor steps is shown. The starting point is a carrier with two spacer units X; a condensation in planar fashion with DNP then took place; a receptor synthesis in spatially resolved fashion (sequences T₁ to T₁₅) then takes place; then either a detection with (a) Cy5-labeled dA15 or (b) with anti-DNP antibodies takes place.

It is thus possible to ascertain the length of the assembled oligonucleotides (T₁-T₁₅) by reaction with the anti-DNP antibodies. This means that this method is suitable for quality control of a receptor synthesis, because the recognition of the probe length and thus also the efficiency of the synthesis which has taken place at this position is possible universally, independently of a sequence, with an antibody. Instead of a control hybridization, for which knowledge of the assembled receptor sequences is a precondition, universal recognition of any number of different sequences is possible via the antibody.

The carrier shown in FIG. 5 with oligo-T receptors can be employed directly for hybridization experiments, for example with fluorescence-labeled dA₁₅ probes. A signal intensity depending on the length of the T probe is obtained.

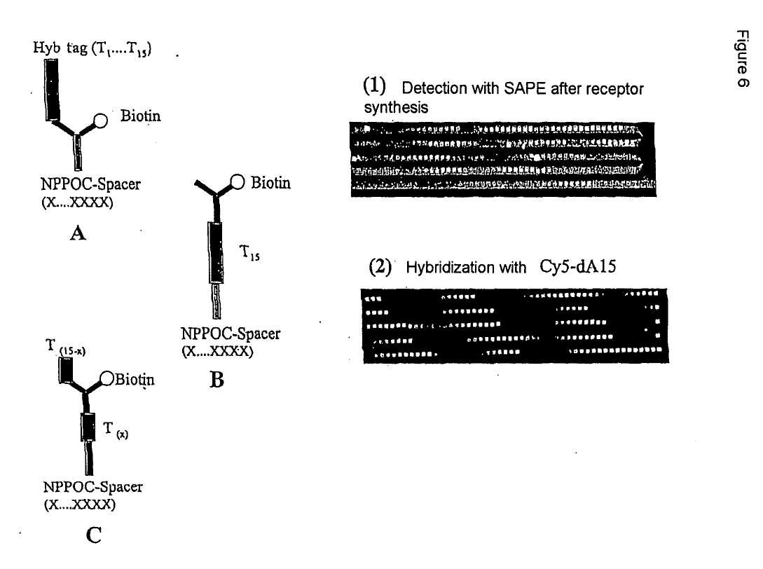

FIG. 6 shows the incorporation of biotin groups into a spacer. Coupling in spatially resolved fashion with 1 to 4 spacer units (X . . . XXXX) was followed by spatially resolved insertion of a biotin group, either:
(A) distally, i.e. before the receptor synthesis,
(B) at any position during the receptor synthesis (in this case between T₁ and T₁₅),
(C) terminally, i.e. after the receptor synthesis is complete.

There are three possibilities for detection:
(1) Detection with a streptavidin-phycoerythrin conjugate (SAPE) after the receptor synthesis is complete

In this case, the length/the success of the spatially resolved synthesis can be inferred directly from the spatially resolved signal intensities. The intensities found show length-dependent behaviour, i.e. the signal emitted after staining with SAPE is higher for a T₁₅ sequence than for a corresponding shorter sequence.
(2) Hybridization with a Cy5-labeled complementary dA15 sequence

In this case, the hybridizations on the complementary thymidine sequences (from T₁₅ . . . T₁) are evident with decreasing signal intensity.

The PE-streptavidin conjugate can be stripped off by washing, e.g. with DMSO/NMI, methanol, ethanol, acetonitrile etc.

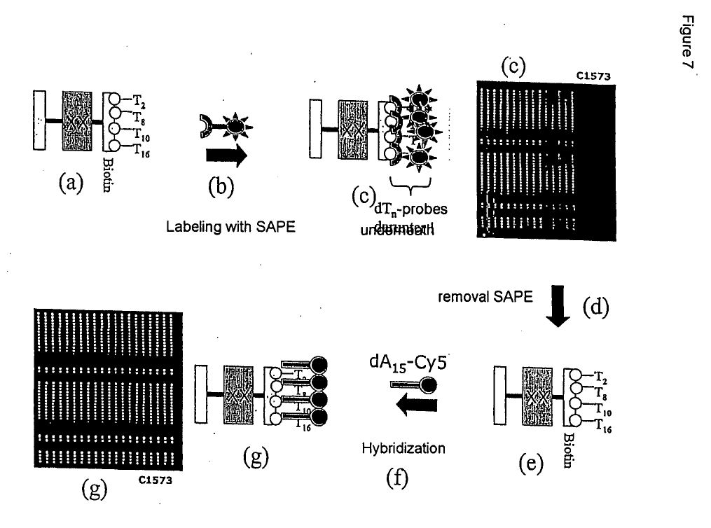

FIG. 7 shows the coupling in planar fashion of two spacer units, the application of a biotin group and a subsequent receptor synthesis.
(a) After the coupling in planar fashion with two spacer units (X), a biotin group was applied in planar fashion. This was followed by a spatially resolved synthesis of homomeric thymidine units (T₂ . . . T₁₆) .
(b) Detection with an SAPE conjugate
(c) Signals are detected in spatially resolved fashion at all positions where thymidine sequences have been assembled in the receptor synthesis. Positive signals are generated. The intensity distribution of the signals follows the density or length of the receptor molecules. This means that the success of the receptor synthesis can be checked even without hybridization immediately after the synthesis.
(d) The SAPE signal-carrying molecules are removed. For this purpose, the DNA chip is washed preferably with an organic solvent (e.g. aceto-nitile, ethanol, DMSO).
(e) The carrier is in its original state without SAPE signal-carrying molecules.
(f) Hybridization with Cy5-labeled complementary dA15 sequence.
(g) In this case, the hybridizations on the complementary thymidine sequences (from T₁₆ . . . T₂) are evident with decreasing signal intensity.

1. A method for producing a carrier for the determination of analytes, comprising the steps:
(a) providing a carrier,
(b) passing liquid with building blocks for synthesizing polymeric receptors over the carrier,
(c) site- or/and time-specifically immobilizing the receptor building blocks on respective predetermined zones on the carrier and
(d) repeating steps (b) and (c) until the desired receptors have been synthesized on the respective predetermined zones,
characterized in that hapten groups are applied to the carrier before, during or/and after the synthesis of the receptors.

2. A method for the quality control of receptor syntheses on a carrier, comprising the steps;
(a) providing a carrier,
(b) applying in planar fashion hapten groups to the carrier surface,
(c) carrying out a receptor synthesis on the carrier,
(d) contacting with a hapten detection reagent which permits detection of hapten groups,
(e) evaluating the hapten group detection on the carrier and
(f) correlating the result of the evaluation with the quality or/and efficiency of the receptorsynthesis.

3. A method for the quality control of receptor syntheses, comprising the steps:
(a) providing a carrier,
(b) carrying out a receptor synthesis on the carrier, with hapten groups being incorporated during the synthesis into the receptor molecules at predeterminedpositions,
(c) contacting with a hapten detection reagent which permits detection of hapten groups,
(d) evaluating the hapten group detection on the carrier and
(e) correlating the result of the evaluation with the quality or/and efficiency of the receptorsynthesis.

4. The method as claimed in claim 1, characterized in that a microfluidic carrier with channels, preferably with closed channels, in which predetermined zones with immobilized receptors are produced is used.

5. The method as claimed in claim 1, characterized in that the receptors are selected from biopolymers such as, for example, nucleic acids, nucleic acid analogs, proteins, peptides and carbohydrates.

6. The method as claimed in claim 1, characterized in that the receptors are selected from nucleic acids and nucleic acid analogs.

7. The method as claimed in claim 1, characterized in that a carrier is produced with aplurality of, preferably with at least 50 and particularly preferably with at least 100,different receptor zones.

8. The method as claimed in claim 1, characterized in that the hapten groups are selected from organic molecules having a molecular weight of up to 2,000, which are recognized by a specific binding partner through a high-affinity interaction.

9. The method as claimed in claim 8, characterized in that the hapten groups are selected from digoxin, digoxigenin, dinitrophenol and biotin or biotin analogs.

10. The method as claimed in claim 1, characterized in that the hapten groups are applied in a planar fashion to the carrier.

11. The method as claimed in claim 1, characterized in that the hapten groups are applied in a site-specific fashion to the carrier.

12. The method as claimed in claim 1, characterized in that the hapten groups are applied directly to the surface of the carrier.

13. The method as claimed in claim 1, characterized in that the hapten groups are inserted into spacer molecules which are disposed between the carrier surface and the receptors.

14. The method as claimed in claim 1, characterized in that the hapten groups are inserted at one or more positions into the receptors synthesized on the carrier.

15. The method as claimed in claim 1, characterized in that the hapten groups are applied reversibly.

16. The method as claimed in claim 1, characterized in that the hapten groups are applied irreversibly.

17. The use of hapten groups for controlling the synthesis of receptors on a carrier.
